(19) United States
(12) Patent Application Publication
(10) Pub. No.: US 2026/0033782 A1
(43) Pub. Date: Feb. 5, 2026

(54) MODULAR PHYSIOLOGICAL SENSOR

(71) Applicant: MASIMO CORPORATION, Irvine, CA (US)

(72) Inventors: Ammar Al-Ali, San Juan Capistrano, CA (US); Kevin Forrest, Rancho Santa Margarita, CA (US)

(21) Appl. No.: 19/357,591

(22) Filed: Oct. 14, 2025

Related U.S. Application Data
(63) Continuation of application No. 18/348,783, filed on Jul. 7, 2023, now Pat. No. 12,465,286.
Application No. 18/348,783 is a continuation of application No. 16/983,357, filed on Aug. 3, 2020, now Pat. No. 11,717,218.
Application No. 16/983,357 is a continuation of application No. 16/182,388, filed on Nov. 6, 2018, now Pat. No. 10,765,367.
Application No. 16/182,388 is a continuation of application No. 14/876,307, filed on Oct. 6, 2015, now Pat. No. 10,154,815.
(60) Provisional application No. 62/061,132, filed on Oct. 7, 2014.

(51) Int. Cl. A61B 5/00; A61B 5/1455; A61B 5/291; A61B 5/369

(52) U.S. Cl. CPC A61B 5/6814; A61B 5/1455; A61B 5/14553; A61B 5/291; A61B 5/369; A61B 5/6833; A61B 5/684; A61B 2560/0443; A61B 2562/04; A61B 2562/06

(57) ABSTRACT

Modular physiological sensors that are physically and/or electrically configured to share a measurement site for the comfort of the patient and/or to ensure proper operation of the sensors without interference from the other sensors. The modular aspect is realized by providing outer housing shapes that generally conform to other physiological sensors; mounting areas for attachment of one sensor to another sensor; providing release liners on the overlapping sensor attachment areas; and/or providing notches, tabs or other mechanical features that provide for the proper placement and interaction of the sensors.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

The present application is a continuation of U.S. patent application Ser. No. 18/348,783 filed Jul. 7, 2023, which is a continuation of U.S. patent application Ser. No. 16/983,357 filed Aug. 3, 2020, now U.S. Pat. No. 11,717,218, which is a continuation of U.S. patent application Ser. No. 16/182,388 filed Nov. 6, 2018, now U.S. Pat. No. 10,765,367, which is a continuation of U.S. patent application Ser. No. 14/876,307 filed Oct. 6, 2015, now U.S. Pat. No. 10,154,815, which claims priority benefit under 35 U.S.C. § 119 (e) to U.S. Provisional Patent Application Ser. No. 62/061,132 filed Oct. 7, 2014, titled Regional Oximetry-EEG Sensor, each of which are hereby incorporated in their entireties by reference herein.

### FIELD OF THE DISCLOSURE

The present disclosure relates to physiological sensors. More specifically, the present disclosure relates to configurations for modular physiological sensors.

### BACKGROUND

Pulse oximetry is a widely accepted noninvasive procedure for measuring the oxygen saturation level of arterial blood, an indicator of a person's oxygen supply. A typical pulse oximetry system utilizes an optical sensor attached to a fingertip to measure the relative volume of oxygenated hemoglobin in pulsatile arterial blood flowing within the fingertip. Oxygen saturation (SpO2), pulse rate and a plethysmograph waveform, which is a visualization of pulsatile blood flow over time, are displayed on a monitor accordingly.

Conventional pulse oximetry assumes that arterial blood is the only pulsatile blood flow in the measurement site. During patient motion, venous blood also moves, which causes errors in conventional pulse oximetry. Advanced pulse oximetry processes the venous blood signal so as to report true arterial oxygen saturation and pulse rate under conditions of patient movement. Advanced pulse oximetry also functions under conditions of low perfusion (small signal amplitude), intense ambient light (artificial or sunlight) and electrosurgical instrument interference, which are scenarios where conventional pulse oximetry tends to fail.

Advanced pulse oximetry is described in at least U.S. Pat. Nos. 6,770,028; 6,658,276; 6,157,850; 6,002,952; 5,769,785 and 5,758,644, which are assigned to Masimo Corporation (“Masimo”) of Irvine, California and are incorporated in their entireties by reference herein. Corresponding low noise optical sensors are disclosed in at least U.S. Pat. Nos. 6,985,764; 6,813,511; 6,792,300; 6,256,523; 6,088,607; 5,782,757 and 5,638,818, which are also assigned to Masimo and are also incorporated in their entireties by reference herein. Advanced pulse oximetry systems including Masimo SET® low noise optical sensors and read through motion pulse oximetry monitors for measuring SpO2, pulse rate (PR) and perfusion index (PI) are available from Masimo. Optical sensors include any of Masimo LNOP®, LNCS®, SofTouch™ and Blue™ adhesive or reusable sensors. Pulse oximetry monitors include any of Masimo Rad 8®, Rad 5®, Rad®-5v or SatShare® monitors.

Advanced blood parameter measurement systems are described in at least U.S. Pat. No. 7,647,083, filed Mar. 1, 2006, titled Multiple Wavelength Sensor Equalization; U.S. Pat. No. 7,729,733, filed Mar. 1, 2006, titled Configurable Physiological Measurement System; U.S. Pat. Pub. No. 2006/0211925, filed Mar. 1, 2006, titled Physiological Parameter Confidence Measure and U.S. Pat. Pub. No. 2006/0238358, filed Mar. 1, 2006, titled Noninvasive Multi-Parameter Patient Monitor, all assigned to Cercacor Laboratories, Inc., Irvine, CA (Cercacor) and all incorporated in their entireties by reference herein. Advanced blood parameter measurement systems include Masimo Rainbow® SET, which provides measurements in addition to SpO2, such as total hemoglobin (SpHb™), oxygen content (SpOC™), methemoglobin (SpMet®), carboxyhemoglobin (SpCO®) and PVI®. Advanced blood parameter sensors include Masimo Rainbow® adhesive, ReSposable™ and reusable sensors. Advanced blood parameter monitors include Masimo Radical-7™, Rad-87™ and Rad-57™ monitors, all available from Masimo. Such advanced pulse oximeters, low noise sensors and advanced blood parameter systems have gained rapid acceptance in a wide variety of medical applications, including surgical wards, intensive care and neonatal units, general wards, home care, physical training, and virtually all types of monitoring scenarios.

### SUMMARY

The present disclosure relates to modular physiological sensors. In some situations in the clinical environment, it is necessary to use multiple physiological sensors in the same general measurement site of a patient. For example, the forehead, arm, hand, ear, and noes are all common areas where multiple physiological sensors may be used at the same time. The present disclosure provides for modular physiological sensors that are physically and/or electrically configured to share the measurement site for the comfort of the patient and to ensure proper operation of the sensors without interference from other sensors. The modular aspect is realized by providing outer housing shapes that generally conform to other physiological sensors; mounting areas for attachment of one sensor to another sensor; providing release liners on the overlapping sensor attachment areas; and/or providing notches, tabs or other mechanical features that provide for the proper placement and interaction of the sensors.

For example, regional oximetry (rO2), also referred to as tissue oximetry and cerebral oximetry, enables the continuous assessment of tissue oxygenation beneath a regional oximetry optical sensor. Regional oximetry helps clinicians detect regional hypoxemia that pulse oximetry alone can miss. In addition, the pulse oximetry capability in regional oximetry sensors can automate a differential analysis of regional to central oxygen saturation. Regional oximetry monitoring is as simple as applying regional oximetry sensors to any of various body sites including the forehead, forearms, chest, upper thigh, upper calf or calf, to name a few. Up to four sensors are connected to a conventional patient monitor via one or two regional oximetry pods. The pods advantageously drive the sensor optics, receive the detected optical signals, perform signal processing on the detected signals to derive regional oximetry parameters and communicate those parameters to a conventional patient monitor through, for example, standard USB ports. Although much of the present disclosure is explained by way of example with respect to EEG and rO2 sensors, it is to be understood that the modular configurations of the sensors can be applied to other types of physiological sensors and are not limited to EEG and rO2 sensors.

In some embodiments, an EEG sensor is advantageously shaped and marked on either side of a connector stem so as to allow regional oximetry (rO2) sensors to be placed in close proximity to the EEG sensor and so as to guide the proper placement of one or more rO2 sensors compactly next to the EEG sensor. The proper placement assistance and joint operation of the sensors provides for improved patient comfort and improved monitoring by ensuring the sensors do not interfere with each other. In some embodiments, the body shape of the EEG sensor is designed to the egg-shaped contours of the rO2 sensor heads. Further, markings on EEG contours correspond to notches on the rO2 sensor heads. These notches allow the rO2 sensor heads to conform to the curvature of a person's forehead. This integrated rO2-EEG sensor combination allows for measuring cerebral regional oximetry in conjunction with EEG parameters, such as depth of consciousness. The EEG sensor is applied first, as the EEG sensor electrodes have particular placement criteria. The EEG sensor markings, as described above, guide placement of the rO2 sensors, as these too require a particular placement for cerebral regional oximetry measurements. The EEG sensor skin-side is advantageously colored black so as to prevent the EEG sensor from reflecting the rO2 sensor-emitted light into the sensor detectors, which would degrade rO2 sensor performance.

In some embodiments, the rO2 sensors connect with a single rO2 pod and cable and the EEG sensor connects with a separate EEG pod and cable. In various other embodiments, a combination rO2-EEG sensor pod houses a single rO2 analog/digital signal processing board and a single EEG signal processing board and the rO2-EEG sensors each connect to the single rO2-EEG sensor pod.

One aspect of a brain analysis sensor is an EEG sensor having a stem, a left branch and a right branch. The left branch and the right branch extend generally perpendicularly from the stem so as to form a branch intersection. A plurality of right and left active electrodes are disposed along the left branch and the right branch. A ground electrode and reference electrode are disposed proximate the branch intersection. A mounting zone is disposed proximate the branch intersection for removable attachment of at least one regional oximetry (rO2) sensor.

In various embodiments, the mounting zone accommodates a regional oximetry sensor head having light emitting and light detecting elements. The mounting zone is marked with a curved line generally indicating a shape of the regional oximetry sensor head. The mounting zone comprises a release layer so that the regional oximetry sensor head removably attaches to the mounting zone. The regional oximetry sensor head has notches that accommodate a curved surface and the mounting zone has notch markings that generally align with the sensor head notches so as to aid regional oximetry sensor placement. The mounting zone is configured to removably attach two regional oximetry sensor heads. A first regional oximetry sensor head is mounted proximate a EEG sensor left branch and a second regional oximetry sensor head is mounted proximate a EEG sensor right branch.

Another aspect of a brain analysis sensor is a sensor method comprising mounting an EEG sensor on a forehead tissue site, mounting a first regional oximetry sensor on the forehead tissue site so as to at least partially overlap a first portion of the EEG sensor and mounting a second regional oximetry sensor on the forehead tissue site so as to at least partially overlap a second portion of the EEG sensor.

In various embodiments, the first portion and the second portion of the EEG sensor are marked for placement of the first and second regional oximetry sensors. A release liner is disposed on the first portion and the second portion for aiding removal of the regional oximetry sensors. The shape of the marked portions conform to shape of the regional oximetry sensors. The marked portions also designate the location of notches on head portions of the regional oximetry sensors.

A further aspect of a brain analysis sensor is an electrical sensor means for passively measuring an EEG signal, an optical sensor means for detecting an oxygen saturation and a placement means for at least partial overlapping the electrical sensor means and the optical sensor means on a tissue site. In an embodiment, the placement means comprises a marking means for designating the partial overlapping. In an embodiment, the marking means comprises at least a partial duplication of the optical sensor means shape on the electrical sensor means.

Regional oximetry sensors and pods are disclosed in U.S. patent application Ser. No. 14/507,620, titled Regional Oximetry Sensor, filed Oct. 6, 2014 by Masimo Corporation, Irvine, CA and incorporated in its entirety by reference herein. An EEG sensor and monitor are disclosed in U.S. patent application Ser. No. 14/470,819, titled Depth of Consciousness Monitor, filed Aug. 27, 2014 by Masimo Corporation, Irvine, CA and incorporated in its entirety by reference herein.

### BRIEF DESCRIPTION OF THE DRAWINGS

FIG. 1 is a perspective view of a brain analysis system having an advantageous modular brain analysis sensor applied to a forehead site and in communications with a physiological monitor for generating simultaneous electroencephalogram (EEG) and left and right forehead regional oximetry (rO2) parameter values and waveforms;

FIGS. 2-3 are perspective views, respectively, of a regional oximetry (rO2) sensor and cable assembly and an EEG sensor and cable assembly;

FIGS. 4A-B are an exploded plan view (FIG. 4A) and a detailed plan view (FIG. 4B), respectively, of a modular brain analysis sensor having an advantageous keyed mounting zone (shaded) for precise, overlaid placement of dual rO2 sensors on an rO2-configured EEG sensor;

FIGS. 5A-E are top, perspective, bottom, side and exploded perspective views, respectively, of an rO2-configured EEG sensor; and

FIGS. 6A-E are top, side, bottom and exploded top perspective views, respectively, of a rO2 sensor and an enlarged perspective view of rO2 sensor optical elements.

### DETAILED DESCRIPTION OF THE PREFERRED EMBODIMENT

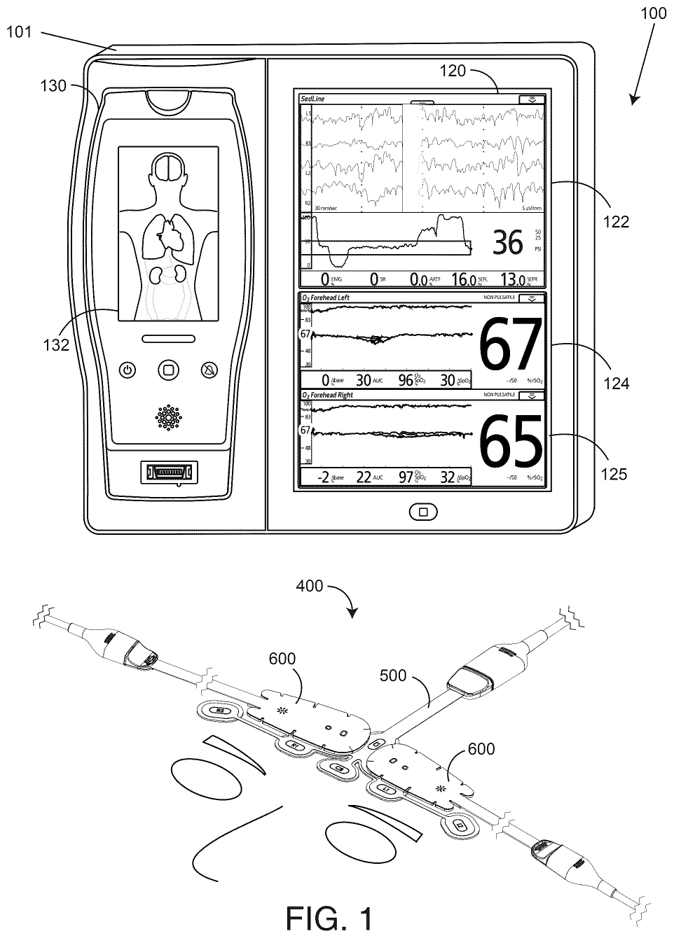

FIG. 1 illustrates a brain analysis system 100 having an advantageous modular brain analysis sensor 400 applied to a forehead tissue site in communications with a physiological monitor 101 for measuring and generating simultaneous electroencephalogram (EEG) and left and right forehead regional oximetry (rO2) parameter values and waveforms. The modular brain analysis sensor 400 can be advantageously assembled and placed within a limited-area forehead site. Also, the rO2 components 600 and EEG component 500 can be advantageously purchased, stocked and used separately and individually, saving hospital and medical care center costs over other, more specialized brain analysis sensors not having separately useable regional oximetry and EEG sensor functions. The same cost savings is realized by modular designs for any and all types of physiological monitoring sensors.

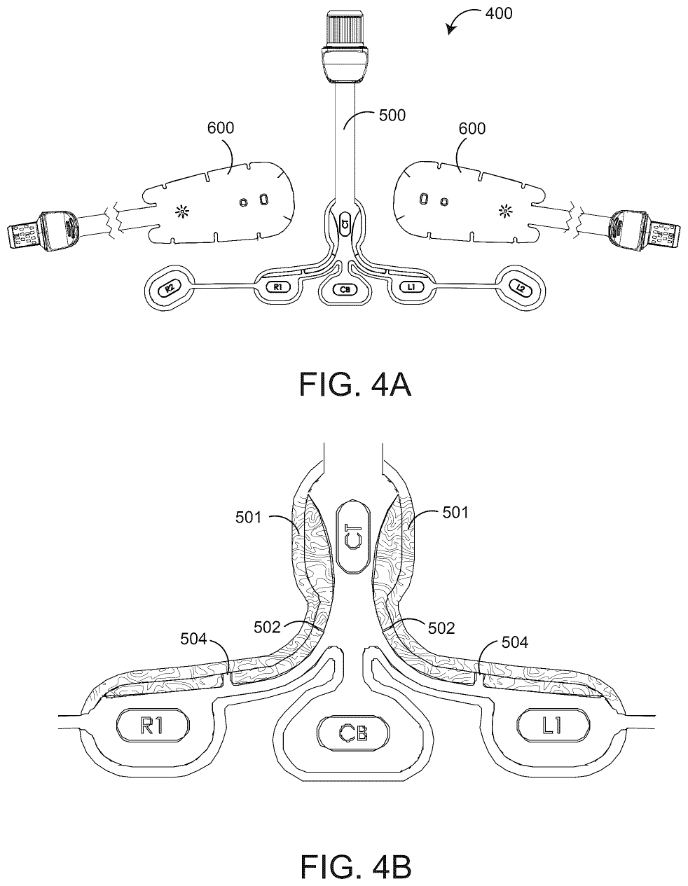

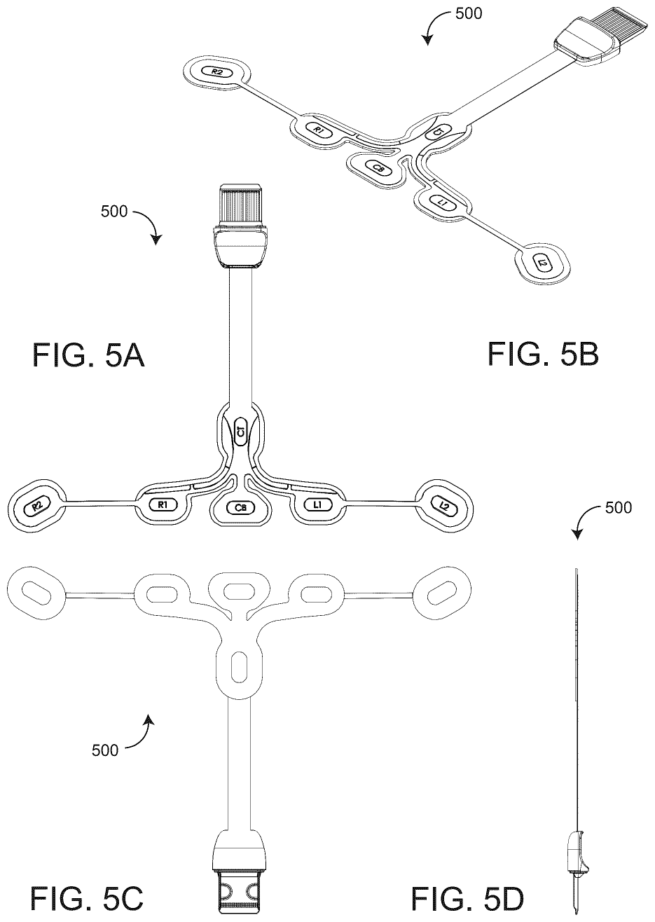

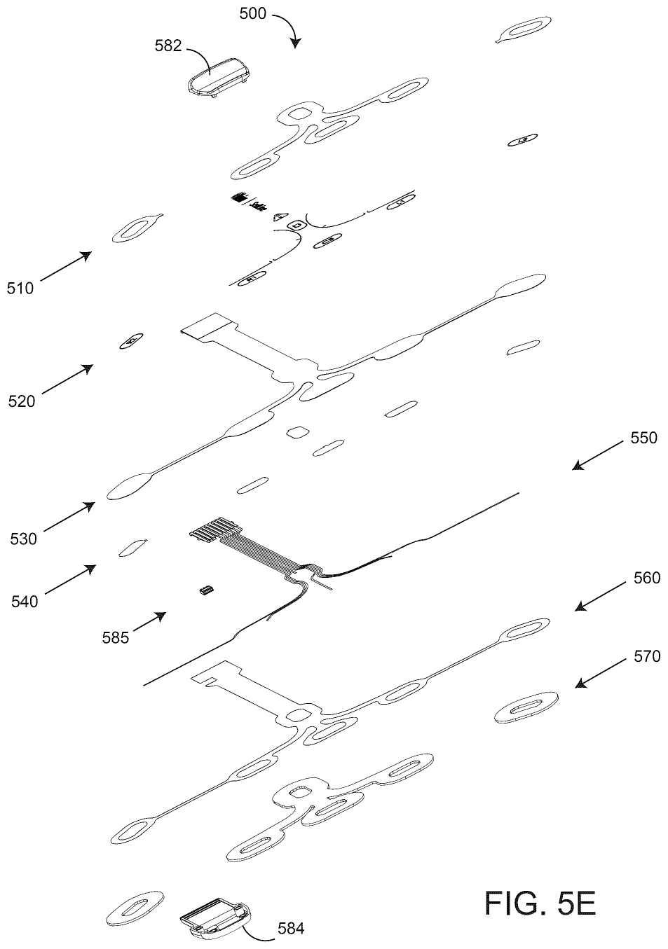

As shown in FIG. 1, the brain analysis sensor 400 has an EEG sensor (FIGS. 4-5) that co-mounts dual regional oximetry (rO2) sensors. Each of these sensor functions are in communications with a physiological monitor 101 having a main display 120 and a (removable) handheld monitor 130 having a handheld display 132. The main display 120 provides EEG waveforms and parameter values 122 in addition to forehead left 124 and forehead right 125 regional oximeter waveforms and parameters. The handheld display 132 provides a 3-D man graphic displaying green, yellow and red organ symbols (brain, lung and kidneys) corresponding to EEG and/or rO2 parameter values. Similar displays can be provided for other physiological parameters as well.

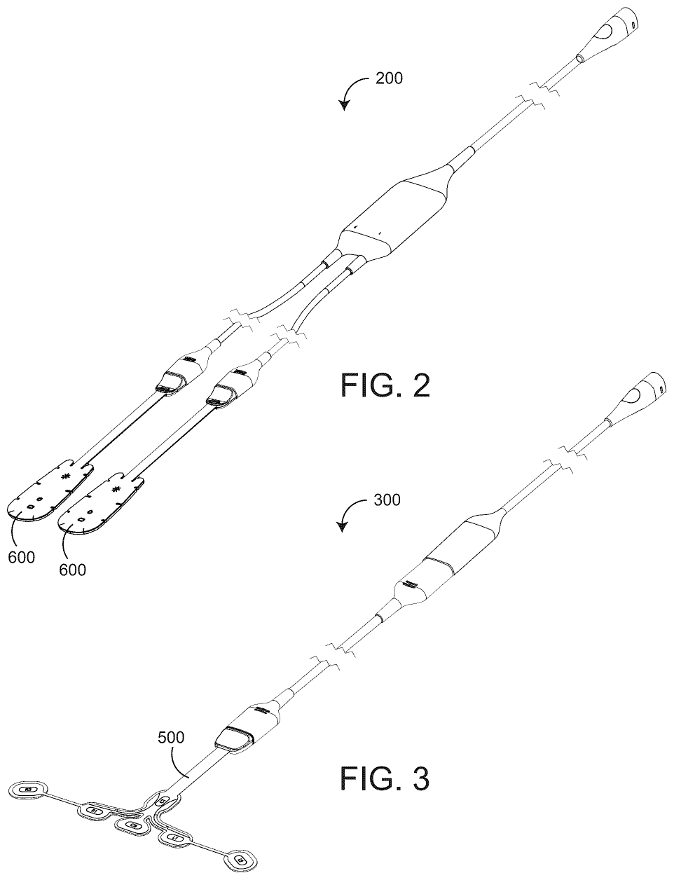

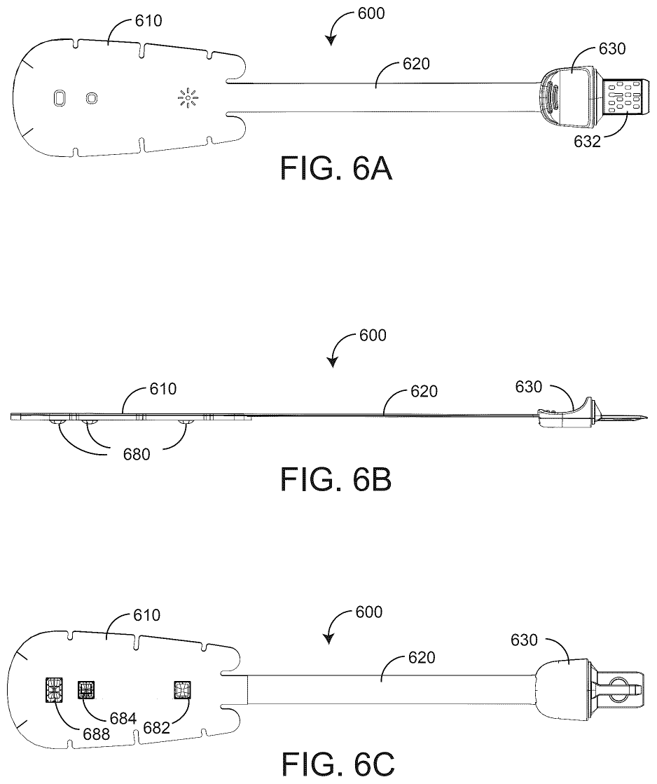

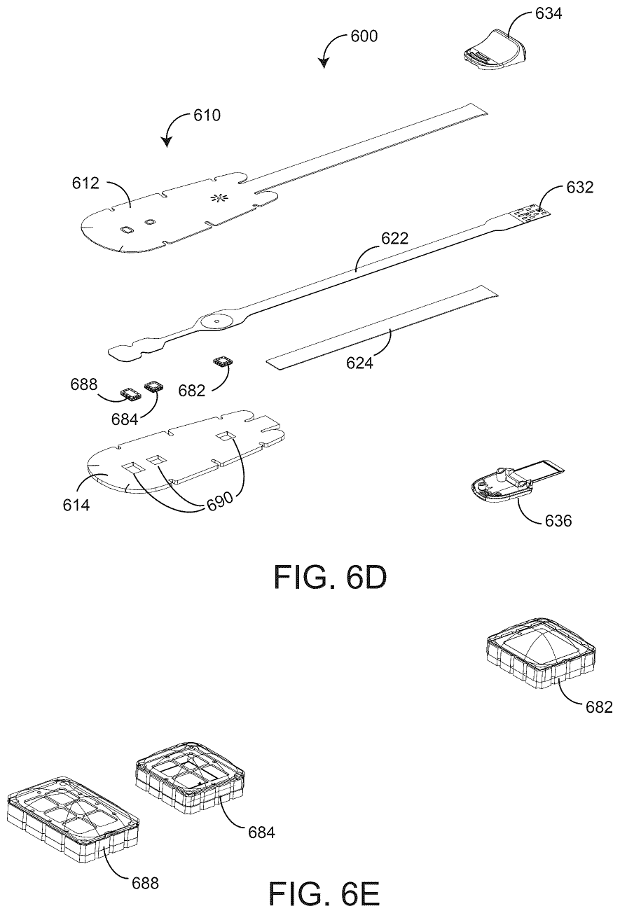

Also shown in FIG. 1, a modular brain analysis sensor 400 advantageously has dual rO2 sensors 600 that overlap right- and left-side portions of a specially-configured and marked (rO2-configured) EEG sensor 500 so as to compactly fit these modular sensors 500, 600 within a limited-space forehead site, as described in detail with respect to FIGS. 2-4, below. An rO2-configured EEG sensor 500 is described in detail with respect to FIGS. 5A-E, below. An regional oximetry sensor 600 is described in detail with respect to FIGS. 6A-E, below.

Further shown in FIG. 1, in an EEG screen portion 122, the physiological monitor 101 display 120 shows 4 simultaneous EEG channels along with a patient state index (PSI) readout versus time so as to enable continuous assessment of both sides of the brain, such as for improved anesthetic management. In addition, forehead left 124 and forehead right 125 regional oximetry waveforms and readouts enable monitoring of brain tissue oxygen saturation and detect regional hypoxemia.

FIGS. 2-3 illustrate, respectively, a regional oximetry (rO2) sensor and cable assembly and an EEG sensor and cable assembly. As shown in FIG. 2, the regional oximetry (rO2) cable assembly 200 interconnects dual rO2 sensors 600 to a physiological monitor 101 (FIG. 1). The rO2 cable assembly has dual sensor connectors at a sensor end, a monitor connector (MOC9) at a monitor end and a rO2 pod mounted between and in communications with the sensor connectors and the monitor connector. Also shown in FIG. 2, the rO2 pod has regional oximetry analog and digital boards. The analog board communicates with one or more of the regional oximetry sensors 600. The digital board enables the pod to perform the sensor communications and signal processing functions of a conventional patient monitor. This allows pod-derived regional oximetry parameters to be displayed on a variety of monitors ranging from simple display devices to complex multiple parameter patient monitoring systems.

As shown in FIG. 3, the EEG cable assembly 300 interconnects an EEG sensor 500 to a physiological monitor 101 (FIG. 1). The EEG cable assembly 300 has an EEG connector at a sensor end, a monitor connector (MOC9) at a monitor end and a EEG pod mounted between and in communications with the sensor connectors and the monitor connector.

FIGS. 4A-B illustrate a modular brain analysis sensor 400 having advantageous keyed mounting zones 501 (shaded) for precise, overlaid placement of dual rO2 sensors on an EEG sensor. In particular, the EEG sensor 500 has two mounting zones 501, one on either side of the interconnected between the EEG electrodes and the EEG sensor connector. Each mounting zone accommodates one of two rO2 sensors (see FIG. 1 and FIG. 4A). Further, each mounting zone 501 (FIG. 4B) is shaped and printed to conform to a top and side portion of an rO2 sensor head 610 (FIGS. 6A-D). Further, each mounting zone has printed notches 502, 504 corresponding to actual notches in the rO2 sensor heads 610 (FIG. 6A) that accommodate curved tissue site surfaces. These printed notches 502, 504 further aid in the alignment of rO2 sensors to the mounting zones 501.

FIGS. 5A-E further illustrate an rO2 configured EEG sensor 500 having a generally “T” shape with six electrodes including two right electrodes R1, R2; two left electrodes L1, L2; a ground electrode CB and a reference electrode CT. As shown in FIG. 5A, the R1, R2, L1, L2 and CB electrodes are disposed across the horizontal top of the “T.” The reference electrode CT is disposed on the vertical middle of the “T.” The advantageous mounting zone 501 (FIG. 4B) is disposed on either side of the vertical middle of the “T” proximate the horizontal top of the “T.”

As shown in FIG. 5E, the EEG sensor 500 has multiple layers including a release liner 510 that allows an attached rO2 sensor 600 (FIG. 1) to be removed and repositioned; artwork 520 including rO2 sensor positioning lines 502 (FIG. 4B); a polyester substrate 530; silver pads 540 (electrodes); silver ink traces 550; a dielectric layer 560 that isolates and protects the traces 550 and a foam pad 570 that contacts a user's skin. The EEG sensor connector includes a top shell 582 and a bottom shell 584. An information element 585 mechanically and electrically connects to the trace layer 550.

FIGS. 6A-E further illustrate a rO2 sensor and its optical elements having a sensor head 610, a stem 620 and a connector 630. The sensor head 610 houses an emitter 682, a near-field detector 684 and a far-field detector 688 within a layered tape having a top side (FIG. 6A) and an adhesive bottom side (FIG. 6C) disposed on a release liner. The release liner is removed so as to adhere the bottom side to a skin surface. The emitter 682 and detectors 684,688 have lens that protrude from the bottom side (FIG. 6E) advantageously providing a robust optics-skin interface. The top side has printed emitter/detector indicators so as to aid precise sensor placement on a patient site. A connector 630 terminates the interconnect 620 at the connector contacts 632.

Also shown in FIG. 6D, a sensor head assembly 610 has a face tape 612, a flex circuit 622, a stem tape 620, a base tape 624, a connector top 634 and a connector base 636. The face tape 612 and base tape 622 encase the flex circuit 622 and corresponding emitter and detectors 682-688.

A modular physiological sensor has been disclosed in detail in connection with various embodiments. These embodiments are disclosed by way of examples only and are not to limit the scope of this disclosure and the claims herein. One of ordinary skill in art will appreciate many variations and modifications. It should be understood specifically that the present mounting zones, tabs, relative shapes and modular configuration can be applied to other physiological sensors including, for example, ear, nose, hand, harm, and/or chest sensors or any other types of physiological sensors where the sensors are configured to jointly measure the same measurement site of a patient.

1. (canceled)

2. A physiological sensor configured for use at a forehead of a subject with a first regional oximetry sensor and a second regional oximetry sensor, the physiological sensor comprising:
a stem comprising an electrical connector, a plurality of stem pads, a ground electrode, and a reference electrode spaced from the ground electrode, each of the ground and reference electrodes operably positioned by a respective one of the plurality of stem pads, the electrical connector configured to be connected to a physiological monitoring device, each of the plurality of stem pads comprising foam;
a left branch connected to and arranged adjacent a first side of the stem, the left branch comprising a plurality of left branch pads and a plurality of left branch electrodes, each of the plurality of left branch electrodes operably positioned by a respective one of the plurality of left branch pads, each of the plurality of left branch pads comprising foam;
a right branch connected to and arranged adjacent a second side of the stem, the right branch comprising a plurality of right branch pads and a plurality of right branch electrodes, each of the plurality of right branch electrodes operably positioned by a respective one of the plurality of right branch pads, each of the plurality of right branch pads comprising foam;
a first mounting indicator configured to indicate mounting of the first regional oximetry sensor onto the physiological sensor such that the first regional oximetry sensor overlaps a first portion of the physiological sensor and skin of the subject's forehead, the first mounting indicator comprising a curved line extending along the left branch and stem and configured to correspond to a shape of a perimeter edge of the first regional oximetry sensor; and
a second mounting indicator configured to indicate mounting of the second regional oximetry sensor onto the physiological sensor such that the second regional oximetry sensor overlaps a second portion of the physiological sensor and the skin of the subject's forehead, the second mounting indicator comprising a curved line extending along the right branch and stem and configured to correspond to a shape of a perimeter edge of the second regional oximetry sensor.

3. The physiological sensor of claim 2, wherein the stem, left branch, and right branch form a T-shape.

4. The physiological sensor of claim 2, wherein the plurality of stem pads are integral with one of the plurality of left branch pads and one of the plurality of right branch pads.

5. The physiological sensor of claim 2, wherein a bottom surface of the physiological sensor is colored black.

6. The physiological sensor of claim 2, further comprising a substrate extending along the stem, left branch, and right branch, wherein the first and second mounting indicators are formed along said substrate.

7. The physiological sensor of claim 6, wherein:
the physiological sensor further comprises:
a first notch arranged on a first portion of the substrate that extends along portions of the left branch and stem; and
a second notch arranged on a second portion of the substrate that extends along portions of the right branch and stem;
the first and second notches are configured to indicate mounting of the first and second regional oximetry sensors onto the first and second portions of the physiological sensor.

8. The physiological sensor of claim 6, further comprising a release liner arranged along a portion of the substrate and configured to allow one or both of the first and second regional oximetry sensors to be removed and repositioned relative to the physiological sensor.

9. The physiological sensor of claim 6, wherein the substrate comprises a different material than the plurality of stem pads, plurality of left branch pads, and plurality of right branch pads.

10. The physiological sensor of claim 6, wherein the plurality of stem pads, plurality of left branch pads, and plurality of right branch pads extend beyond the substrate.

11. An assembly comprising the physiological sensor of claim 2 and the first and second regional oximetry sensors, wherein each of the first and second regional oximetry sensors comprise a plurality of detectors and at least one emitter.

12. The physiological sensor of claim 2, wherein:
the plurality of left branch pads comprises two left branch pads;
the plurality of left branch electrodes two electrodes;
the plurality of right branch pads comprises two right branch pads; and
the plurality of right branch electrodes two electrodes.

13. A physiological sensor configured for use at a forehead of a subject with a first regional oximetry sensor and a second regional oximetry sensor, the physiological sensor comprising:
a stem comprising an electrical connector, a ground electrode, and a reference electrode, the electrical connector configured to be connected to a physiological monitoring device;
a left branch connected to and arranged adjacent a first side of the stem, the left branch comprising at least one left branch electrode;
a right branch connected to and arranged adjacent a second side of the stem, the right branch comprising at least one right branch electrode;
a first mounting indicator comprising a curved line extending along the left branch and stem and configured to correspond to a shape of a portion of the first regional oximetry sensor; and
a second mounting indicator comprising a curved line extending along the right branch and stem and configured to correspond to a shape of a portion of the second regional oximetry sensor.

14. The physiological sensor of claim 13, wherein the stem, left branch, and right branch form a T-shape.

15. The physiological sensor of claim 13, further comprising a substrate extending along the stem, left branch, and right branch, wherein the first and second mounting indicators are formed along said substrate.

16. The physiological sensor of claim 15, further comprising a release liner arranged along a portion of the substrate and configured to allow one or both of the first and second regional oximetry sensors to be removed and repositioned relative to the physiological sensor.

17. The physiological sensor of claim 13, further comprising:
a first stem pad for operably positioning the ground electrode;
a second stem pad for operably positioning the reference electrode;
at least one left branch pad for operably positioning the at least one left branch electrode; and
at least one right branch pad for operably positioning the at least one right branch electrode.

18. The physiological sensor of claim 17, wherein:
the left branch comprises a first left branch electrode, a second left branch electrode, a first left branch pad for operably positioning the first left branch electrode, and a second left branch pad for operably positioning the second left branch electrode; and
the right branch comprises a first right branch electrode, a second right branch electrode, a first right branch pad for operably positioning the right left branch electrode, and a second right branch pad for operably positioning the second right branch electrode.

19. The physiological sensor of claim 18, wherein the first stem pad, second stem pad, first left branch pad, and first right branch pad are integrally formed from a foam material.

20. The physiological sensor of claim 17, wherein the physiological sensor further comprises a substrate extending along the stem, left branch, and right branch, and wherein the substrate comprises a different material than each of the first stem pad, second stem pad, at least one left branch pad, and at least one right branch pad.

21. An assembly comprising the physiological sensor of claim 13 and the first and second regional oximetry sensors, wherein each of the first and second regional oximetry sensors comprise a plurality of detectors and at least one emitter.
